(19) United States
(12) Patent Application Publication
(10) Pub. No.: US 2025/0029706 A1
(43) Pub. Date: Jan. 23, 2025

(54) INFORMATION PROCESSING APPARATUS, INFORMATION PROCESSING METHOD, AND STORAGE MEDIUM STORING PROGRAM

(71) Applicant: CANON KABUSHIKI KAISHA, Tokyo (JP)

(72) Inventor: HIROFUMI KUBOTA, Kanagawa (JP)

(21) Appl. No.: 18/778,776

(22) Filed: Jul. 19, 2024

(30) Foreign Application Priority Data
Jul. 21, 2023 (JP) 2023-119162

(51) Int. Cl. G16H 30/20; G06F 3/14; G06T 7/00

(52) U.S. Cl. CPC G16H 30/20; G06F 3/14; G06T 7/0002; G06T 2207/30168

(57) ABSTRACT

An information processing apparatus includes at least one memory storing instructions and at least one processor. The at least one processor is configured to execute the instructions to generate a first integrated image by integrating a plurality of radiation images obtained by image capturing using a radiation generating apparatus that generates radiation and a radiation imaging apparatus, and generate a second integrated image different from the first integrated image using the plurality of radiation images and at least one radiation image obtained by the image capturing after the plurality of radiation images are obtained; and control a display device to change a display based on a determination regarding an image quality of the first integrated image to a display based on a determination regarding an image quality of the second integrated image.

### BACKGROUND

### Field of the Disclosure

The present disclosure relates to an information processing apparatus, an information processing method, and a storage medium storing a program.

### Description of the Related Art

As an image capturing apparatus used for non-destructive inspection using an X-ray, a radiation imaging apparatus that has a flat panel detector (abbreviated as an FPD) formed of a semiconductor material is widely used. In a non-destructive inspection, an integrated image obtained by integrating a plurality of radiation images is used to reduce a quantum noise component. In addition, the integrated image used in the non-destructive inspection needs to satisfy the defined signal-to-noise ratio (SNR) (the Japanese Industrial Standards: JIS Z 31110: 2017).

Japanese Patent Laid-Open No. 2019-058608 discloses a method of obtaining and displaying the SNR of a radiation image after completion of image capturing.

In the method disclosed in Japanese Patent Laid-Open No. 2019-058608, for example, in a case where it is found that an image does not satisfies the defined SNR after image capturing, it may be necessary to perform image re-capturing. In a case where it is necessary to perform image re-capturing, the efficiency of an inspection decreases.

### SUMMARY

An object of the present disclosure is to suppress the need for image re-capturing.

An information processing apparatus according to some embodiments of the present disclosure includes at least one memory storing instructions and at least one processor. The at least one processor is configured to execute the instructions to generate a first integrated image by integrating a plurality of radiation images obtained by image capturing using a radiation generating apparatus that generates radiation and a radiation imaging apparatus, and generate a second integrated image different from the first integrated image using the plurality of radiation images and at least one radiation image obtained by the image capturing after the plurality of radiation images are obtained; and control a display unit to change a display based on a determination regarding an image quality of the first integrated image to a display based on a determination regarding an image quality of the second integrated image.

Further features of the various embodiments will become apparent from the following description of exemplary embodiments with reference to the attached drawings.

### BRIEF DESCRIPTION OF THE DRAWINGS

FIG. 1 is a diagram illustrating an example of a configuration of an information processing system according to the present disclosure.

FIG. 2 is a flowchart of an operation of a computer according to the present disclosure.

FIG. 3 is a diagram illustrating an example of a display on a display unit according to the present disclosure.

FIG. 4 is a diagram illustrating an example of a display on the display unit according to the present disclosure.

FIG. 5 is a flowchart of an operation of the computer according to the present disclosure.

FIG. 6 is a diagram illustrating an example of a display on the display unit according to the present disclosure.

FIG. 7 is a diagram illustrating an example of a display on the display unit according to the present disclosure.

FIG. 8 is a diagram illustrating an example of a display on the display unit according to the present disclosure.

### DESCRIPTION OF THE EMBODIMENTS

Embodiments will be described below with reference to the accompanying drawings. The following embodiments do not limit the claims herein. Although a plurality of features are described in the embodiments, not all of the plurality of features may be essential for implementing the present disclosure, and the plurality of features may be combined in any manner. In the accompanying drawings, configurations that are the same or similar are denoted by the same reference signs, and redundant description is omitted.

### First Embodiment

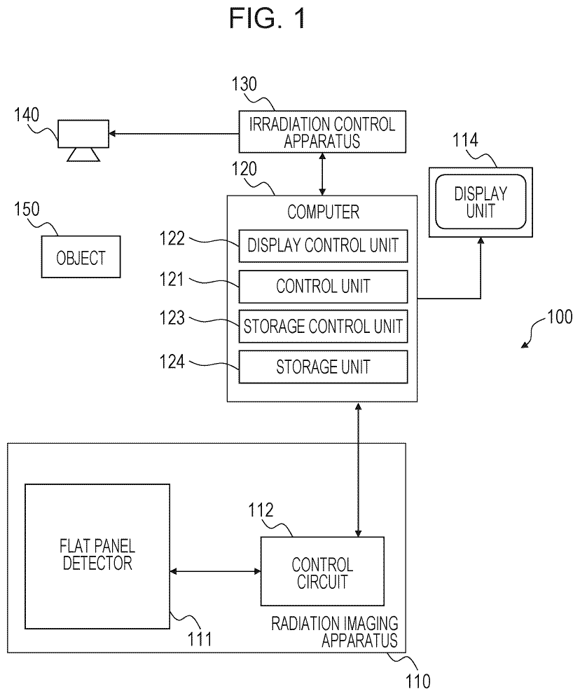

FIG. 1 illustrates an example of a configuration of an information processing system 100 according to a first embodiment of the present disclosure. The information processing system 100 is configured to electrically capture an optical image formed by radiation and obtain an electrical radiation image. The radiation is typically an X-ray, but may be an α-ray, a β-ray, or a γ-ray. The information processing system 100 includes, for example, a radiation imaging apparatus 110, a computer 120 as an information processing apparatus, a display unit 114, an irradiation control apparatus 130, and a radiation generating apparatus 140.

The radiation generating apparatus 140 starts to perform irradiation with radiation in accordance with an irradiation command (radiation command) from the irradiation control apparatus 130. The radiation emitted from the radiation generating apparatus 140 passes through an object 150 and enters the radiation imaging apparatus 110. The radiation generating apparatus 140 stops the irradiation with the radiation in accordance with a stop command from the irradiation control apparatus 130.

The radiation imaging apparatus 110 is an image capturing apparatus that outputs a radiation image based on the radiation. The radiation imaging apparatus 110 includes a flat panel detector 111 and a control circuit 112. The flat panel detector 111 generates an image signal based on the radiation that has entered the radiation imaging apparatus 110.

The image signal is data indicating the radiation image. The control circuit 112 controls an operation of the flat panel detector 111. Also, the control circuit 112 may generate, based on the image signal received from the flat panel detector 111, a stop signal for stopping irradiation with radiation from the radiation generating apparatus 140. The stop signal is supplied to the irradiation control apparatus 130. The irradiation control apparatus 130 transmits a stop command to the radiation generating apparatus 140 in response to the stop signal. The control circuit 112 may be a programmable logic device (PLD) such as a field programmable gate array (FPGA), or a dedicated circuit such as an application specific integrated circuit (ASIC). Also, the control circuit 112 may be formed by combining a general-purpose processing circuit such as a processor and a storage circuit such as a memory. In this case, functions of the control circuit 112 may be implemented by the general-purpose processing circuit executing a program stored in the storage circuit.

In a non-destructive inspection which is performed without destroying an object not to be inspected, the irradiation control apparatus 130 and the computer 120 may not be connected to each other for synchronization. In this case, a user directly operates the irradiation control apparatus 130 to stop irradiation with radiation. The object not to be inspected is, for example, a printed board or piping. For example, in the inspection of the piping, whether the inside and outside of the piping are damaged, a degraded state of a material forming the piping, and the like are inspected.

The computer 120 as the information processing apparatus is connected to the irradiation control apparatus 130 and the radiation imaging apparatus 110 such that the computer 120 can communicate with the irradiation control apparatus 130 and the radiation imaging apparatus 110. The communication includes direct communication between the computer 120, and the irradiation control apparatus 130 and the radiation imaging apparatus 110, and communication via the other apparatus. The communication via the other apparatus indicates, for example, a mode in which the computer 120 communicates with the radiation generating apparatus 140 via the irradiation control apparatus 130. The computer 120 includes an irradiation controller (not illustrated), a receiver (not illustrated), and a signal processor (not illustrated). The irradiation controller controls the radiation imaging apparatus 110 and the irradiation control apparatus 130. The receiver receives a radiation image from the radiation imaging apparatus 110. The signal processor processes the radiation image obtained by the radiation imaging apparatus 110.

The computer 120 further includes a control unit 121 and a display control unit 122.

The control unit 121 generates an integrated image by integrating a plurality of radiation images and makes a determination regarding the image quality of the integrated image. The integrating of the plurality of radiation images is, in other words, the addition of the plurality of radiation images. In addition, adding a new radiation image to an integrated image obtained by integrating a plurality of radiation images is also referred to as integrating of the plurality of radiation images. Further, the determination regarding the image quality of the integrated image is, for example, a determination as to whether the signal-to-noise ratio (SNR) of the integrated image satisfies a predetermined value. The SNR is the ratio of a signal component of the integrated image to a noise component of the integrated image. The control unit 121 obtains the SNR of the integrated image and compares the SNR with the predetermined value to determine whether the SNR of the integrated image satisfies the predetermined value.

The display control unit 122 controls the display unit 114 to display a display based on the determination by the control unit 121. In this case, in a case where the SNR of the integrated image satisfies the predetermined value, the display based on the determination is a display prompting to end image capturing, such as “Please stop irradiation with radiation.”, “Please stop the operation of the radiation imaging apparatus.”, or “The inspected image has been properly stored.”. The display prompting to end the image capturing may be a display of information such as “Please press the end image capturing button.”, “The inspection has been completed.”, or “The SNR has reached the predetermined value.”. In addition, the display based on the determination may be a display of an SNR value, such as “SNR: 70”. In a case where the SNR of the integrated image does not satisfy the predetermined value, the display based on the determination may be a display of information such as “Please continue irradiation with radiation.”, or “The SNR has not reached the predetermined value”. The display control unit 122 controls the display unit 114 to display at least one of the above-described displays based on the determination regarding the image quality of the integrated image. These displays are examples of the display based on the determination regarding the image quality of the integrated image.

The computer 120 further includes a storage unit 124 and a storage control unit 123. The storage unit 124 is, for example, a hard disk, a flash memory, or the like. The storage control unit 123 controls to store the integrated image generated by the control unit 121 to the storage unit 124.

Each of the irradiation controller, the receiver, and the signal processor may be a dedicated circuit or may be formed by combining a general-purpose processing circuit and a storage circuit in a similar manner to the control circuit 112. As an example, the irradiation control apparatus 130 includes an irradiation switch. When a user turns on the irradiation switch, the irradiation control apparatus 130 transmits an irradiation command to the radiation generating apparatus 140 and transmits a start notification indicating the start of irradiation with radiation to the computer 120. The computer 120 that has received the start notification notifies the control circuit 112 of the radiation imaging apparatus 110 of the start of irradiation with radiation in response to the start notification. The control unit 121 may not be included in the computer 120 and may be included in the radiation imaging apparatus 110.

In a case where the irradiation control apparatus 130 and the computer 120 are not synchronously connected to each other, the flat panel detector 111 continues an image read operation and detects the start of irradiation with radiation from an output value of the image signal.

The display unit 114 is, for example, a liquid crystal monitor. The display unit 114 is controlled by the display control unit 122. The display unit 114 displays a radiation image, an integrated image, various notifications (irradiation stop notification and the like) to an operator, an SNR, and the like. The display unit 114 may be separated from the computer 120 or may be included in the computer 120.

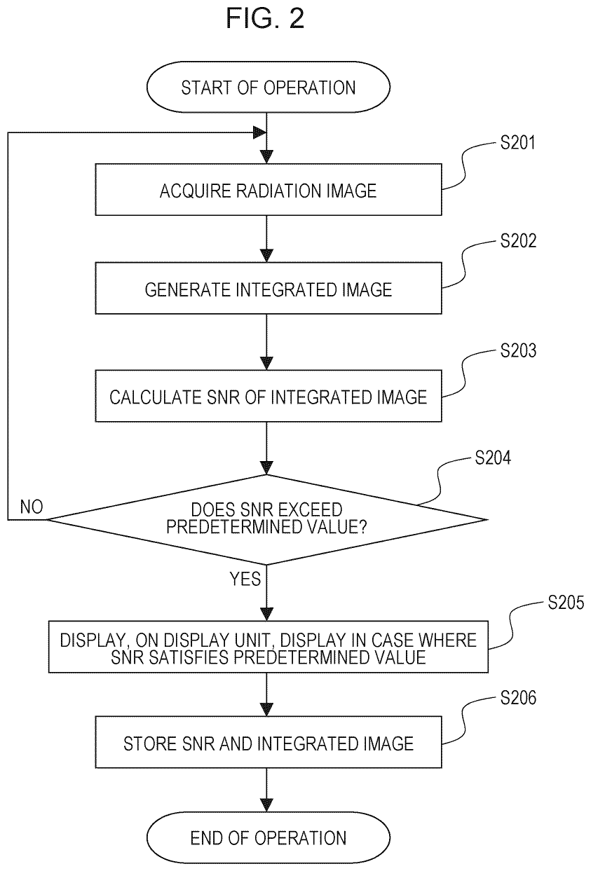

Next, a procedure of operation of the computer 120 will be described with reference to FIG. 2. FIG. 2 illustrates an example of the procedure from acquisition of a radiation image to displaying of the stop of irradiation on the display unit 114. In this case, an operation of the information processing system 100 is controlled by the computer 120. An operation of the radiation imaging apparatus 110 is controlled by the control circuit 112 under control by the computer 120. For example, the operation illustrated in FIG. 2 is started with the operation of the radiation imaging apparatus 110.

The “operation” illustrated in FIG. 2 indicates the operation of the computer 120.

When the operation is started, the control unit 121 included in the computer 120 acquires a radiation image from the radiation imaging apparatus 110 in S201. The acquired radiation image is stored in the storage unit 124 included in the computer 120. In this case, the acquired radiation image may be one image or a plurality of images continuously captured.

Next, in S202, the computer 120 generates an integrated image using the radiation image acquired in S201. The display control unit 122 included in the computer 120 controls the display unit 114 to display the generated integrated image. The generated integrated image is an example of a first integrated image. The display control unit 122 may control the display unit 114 to display the plurality of radiation images continuously captured as a moving image, instead of displaying the generated integrated image. The displaying of the images as the moving image indicates displaying of the plurality of captured radiation images at a predetermined time interval (for example, 1 second) in the order that the radiation images have been captured. The predetermined time interval may be set to any value or may be a time interval (frame rate) at which the image capturing has been performed. The display control unit 122 may control the display unit 114 to display both of the integrated image and the moving image.

Next, in S203, the computer 120 calculates an SNR of the integrated image. The computer 120 calculates the SNR in a predetermined region in the integrated image. The predetermined region may be selected and determined by the operator. Also, the computer 120 may analyze the integrated image and determine the predetermined region. A method of calculating the SNR is not limited. For example, the computer 120 obtains the amount of noise by subtracting two temporarily consecutive captured images of the same object from one another and dividing the result of the subtraction by the square root of 2. In this case, as the two images, the most recent two radiation images may be used. Subsequently, the computer 120 calculates the SNR by dividing the amount of a signal of the integrated image by the obtained amount of noise.

Next, in S204, the computer 120 compares the calculated SNR with the predetermined value. The predetermined value provides an image quality required as an image to be inspected. The predetermined value is, for example, “70” or the like. For example, the predetermined value is defined in the Japanese Industrial Standards (JIS Z 3110: 2017). In the present embodiment, the predetermined value is set in the computer 120 in advance. However, the operator may change the set predetermined value. In a case where the SNR does not exceed the predetermined value (S204: NO) as a result of the comparison between the SNR and the predetermined value, the computer 120 acquires a new radiation image (S201). Subsequently, the computer 120 integrates the acquired new radiation image with the integrated image to generate a new integrated image (S202). The computer 120 may integrate the new radiation image with the plurality of radiation images used for generating the integrated image, instead of integrating the acquired new radiation image with the integrated image. In addition, every time the computer 120 acquires a single new radiation image, the computer 120 may integrate the acquired new radiation image with the integrated image. Every time the computer 120 continuously acquires three new radiation images, the computer 120 may integrate only the third radiation image among the three new radiation images with the integrated image. The new integrated image is an example of a second integrated image. Then, the display control unit 122 controls the display unit 114 to display the new integrated image.

In this case, in a case where an already displayed integrated image is present, the displayed integrated image is updated to the new integrated image. The computer 120 calculates an SNR of the new integrated image (S203). Next, the computer 120 compares the calculated SNR of the new integrated image with the predetermined value (S204).

That is, in a case where the SNR does not exceed the predetermined value (S204: NO), the computer 120 repeats the processing in S201 to S204 to repeat generation of a new integrated image. That is, in a case where the SNR of the second integrated image does not exceed the predetermined value, the computer 120 generates a third integrated image newer than the second integrated image. By repeating generation of a new integrated image, an integrated image can be obtained by integrating a larger number of radiation images. The larger the number of radiation images integrated into an integrated image, the higher the SNR of the integrated image.

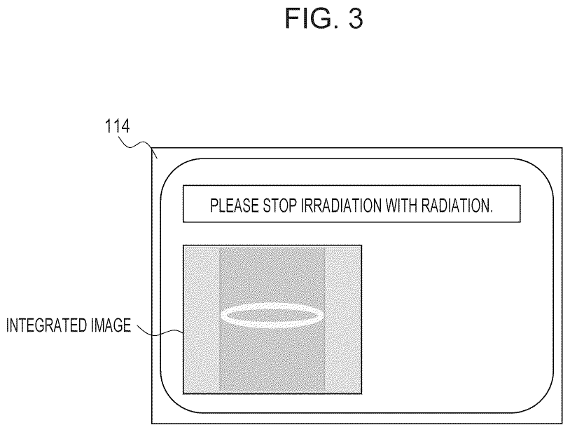

In a case where the calculated SNR exceeds the predetermined value (S204: YES) as a result of the comparison between the calculated SNR and the predetermined value, the computer 120 determines that the integrated image satisfies the required image quality, and displays, on the display unit 114, a display (e.g., a display prompting to end image capturing as described above) for the SNR that satisfies the predetermined value (S205). FIG. 3 illustrates an example in which the display prompting to end the image capturing is displayed. In a case where a display for the SNR that does not satisfy the predetermined value is displayed before the display for the SNR that satisfies the predetermined value is displayed, the computer 120 changes the display to the display for the SNR that satisfies the predetermined value.

Subsequently, the computer 120 stores the calculated SNR and the integrated image in the storage unit 124 included in the computer 120 (S206). After the storing, the computer 120 ends the operation. The storage unit 124 is, for example, a hard disk, a flash memory, or the like.

The procedure of the operation of the computer 120 according to the first embodiment is described above.

Since the procedure of the operation described above is provided, the operator can use the display unit 114 to check whether the integrated image satisfies a desired image quality during image capturing. Therefore, the operator can stop irradiation with radiation after checking the integrated image satisfying the desired image quality. That is, unlike the existing techniques, it is not necessary to check whether an integrated image satisfies the desired image quality after the end of image capturing, and it is possible to reduce image re-capturing.

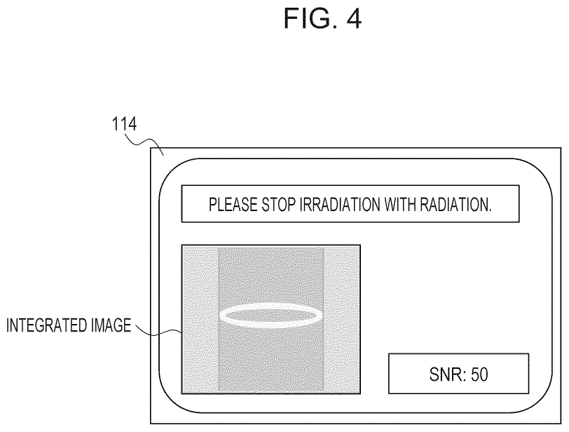

The display control unit 122 included in the computer 120 may control the display unit 114 to further display the SNR of the integrated image in addition to the display prompting to end image capturing, such as “Please stop irradiation with radiation.”, as illustrated in FIG. 4. The SNR of the integrated image may be displayed only in a case where the SNR of the integrated image exceeds the predetermined value (in a case where the integrated image satisfies the desired image quality). The SNR of the integrated image may be displayed regardless of whether the SNR of the integrated image exceeds the predetermined value.

In a case where the SNR of the integrated image is displayed regardless of whether the SNR of the integrated image exceeds the predetermined value, the operator can check the SNR of the integrated image during image capturing. Therefore, the operator can stop irradiation with radiation after checking the SNR of a radiation image reaching the predetermined value.

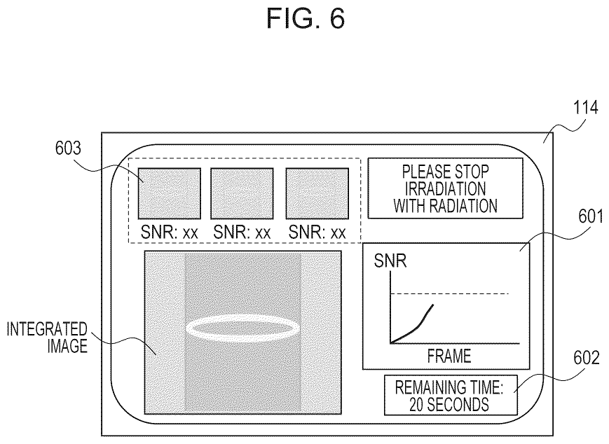

In addition, as illustrated in FIG. 6, the display control unit 122 included in the computer 120 may control the display unit 114 to further display a graph 601 indicating a change in the SNR of the integrated image. The graph 601 is, for example, a graph in which the horizontal axis indicates the number of frames and the vertical axis indicates the SNR of the integrated image. By displaying the graph 601, the operator can grasp a time (remaining time) required for the SNR of the integrated image to reach the predetermined value. The horizontal axis of the graph 601 may indicate time. That is, the graph 601 may be any graph as long as the graph indicates a change in the SNR of the integrated image. Therefore, for example, instead of the graph 601, a table format in which the SNR of the integrated image and time are indicated may be used.

In addition, the display control unit 122 included in the computer 120 may control the display unit 114 to further display a time (remaining time 602) required for the SNR of the integrated image to reach the predetermined value, as illustrated in FIG. 6. The control unit 121 included in the computer 120 calculates, based on the change in the SNR of the integrated image, the remaining time 602 until the SNR of the integrated image reaches the predetermined value. Then, the display control unit 122 controls the display unit 114 to further display the calculated remaining time 602. A method of calculating the remaining time 602 is, for example, to calculate the SNR of a new integrated image when the new integrated image is generated, compare the calculated SNR with the SNR of an integrated image before the generation of the new integrated image, calculate a change rate of the SNR of the integrated image, and calculate the remaining time 602 based on the calculated change rate. The display control unit 122 may control the display unit 114 to display the number of remaining images to be captured, instead of the remaining time 602. That is, the display control unit 122 may control the display unit 114 to display any display as long as the operator can grasp the time (remaining time) required for the SNR of the integrated image to reach the predetermined value.

In addition, in a case that the control unit 121 included in the computer 120 determines that the SNR of the integrated image clearly does not reach the predetermined value, the display control unit 122 included in the computer 120 may control the display unit 114 to display a warning. The case where “the SNR of the integrated image clearly does not reach the predetermined value” is, for example, a case where the SNR does not increase even when a new integrated image is repeatedly generated by repeating the processing in S201 to S204. In other words, the case where “the SNR of the integrated image clearly does not reach the predetermined value” is a case where the change rate of the SNR of the new integrated image is smaller than a change rate threshold set in advance. The change rate threshold is set to, for example, “10%” or the like. In a case where “10%” is set as the change rate threshold, for example, in a case where the ratio of the SNR2 of a new integrated image (second integrated image) to the SNR1 of an integrated image (first integrated image) immediately before the generation of the new integrated image, that is, SNR2/SNR1, is less than 110%, the warning is displayed. The threshold for the determination as to whether “the SNR clearly does not reach the predetermination value” may be, for example, the value of the SNR itself.

In a case where the threshold is the value of the SNR itself, for example, in a case where the difference between the SNR2 of the new integrated image (second integrated image) and the SNR1 of the integrated image (first integrated image) immediately before the generation of the new integrated image, that is, SNR2−SNR1 is “less than 3”, the warning is displayed. The above-described “10%” and “less than 3” are examples of a first threshold.

Also, for example, the case where “the SNR clearly does not reach the predetermined value” may be a case where an estimated remaining time is equal to or longer than a predetermined time (e.g., 30 minutes). The predetermined time may be set by the operator. This is due to the fact that the time until the SNR reaches the predetermined value may largely vary depending on an object to be inspected. The estimated remaining time is an example of time information. The predetermined time is an example of a second threshold.

The display control unit 122 included in the computer 120 may control the display unit 114 to further display each of radiation images used for generating an integrated image, as illustrated in FIG. 6. FIG. 6 illustrates an example in which the most recent three acquired radiation images 603 are displayed.

The display control unit 122 included in the computer 120 may control the display unit 114 to further display each of the SNRs of the radiation images used for generating the integrated image. This display may be a graph or a display in a table format (list format).

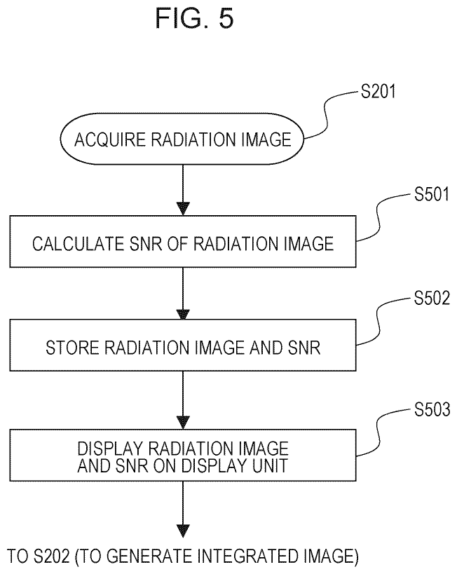

FIG. 5 illustrates a procedure of operation from acquisition of radiation images by the computer 120 to displaying of the SNRs of the radiation images on the display unit 114 by the computer 120.

The computer 120 starts the operation by acquiring the radiation images (S201). S201 illustrated in FIG. 5 is the same as S201 illustrated in FIG. 2.

After S201, the computer 120 calculates SNRs of the radiation images (S501). A method of calculating the SNRs may be the same as or similar to that in S203 (the calculation of the SNR of an integrated image) illustrated in FIG. 2.

After S501, the computer 120 stores the radiation images and the SNRs in the storage unit 124 included in the computer 120 (S502).

After S502, the computer 120 displays the radiation images and the SNRs on the display unit 114 (S503).

After displaying the radiation images and the SNRs on the display unit 114, the computer 120 performs the processing in S202 and the subsequent processing illustrated in FIG. 2.

Since each of the radiation images used for generating the integrated image and each of the SNRs are displayed in the above-described manner, the operator can check whether the dosage of radiation is stable. Therefore, from the aspect of the stability of the dosage of the radiation, it is possible to grasp whether an integrated image having a desired image quality can be obtained in current image capturing. As a result, the operator can quickly determine whether image re-capturing needs to be performed, and a more effective inspection can be performed.

In addition, in a case where the irradiation control apparatus 130 and the computer 120 are synchronized with each other, the computer 120 may output a signal based on a determination regarding the image quality. For example, in a case where the SNR of the integrated image exceeds the predetermined value, the computer 120 may output an irradiation stop signal to the irradiation control apparatus 130. By outputting the irradiation stop signal, it is possible to stop irradiation with radiation without a manual operation by the operator. The irradiation stop signal is transmitted and received via communication by an output unit (not illustrated) included in the computer 120 and the irradiation control apparatus 130. For example, the output unit may be an antenna for wireless communication or may be a LAN terminal for wired communication.

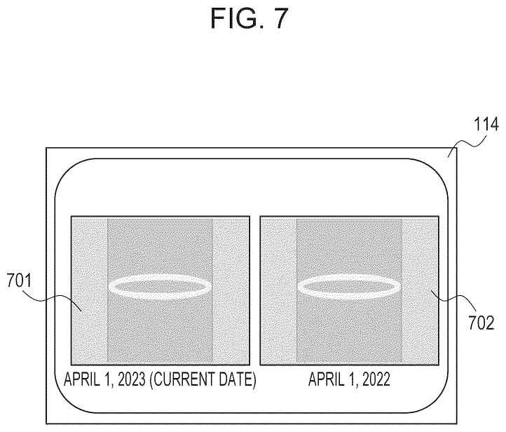

The display control unit 122 included in the computer 120 may control the display unit 114 to display an integrated image 701 and an integrated image 702 side by side, as illustrated in FIG. 7. The integrated image 702 is obtained in image capturing at a different time from a time when the integrated image 701 is obtained. FIG. 7 illustrates an example in which the integrated image 701 obtained in image capturing on Apr. 1, 2023 (current date) and the integrated image 702 obtained in image capturing on Apr. 1, 2022 (past date) are displayed side by side. The integrated image 702 is an example of a fourth integrated image. Since the integrated image 701 and the integrated image 702 are displayed side by side, the operator can easily compare the current integrated image with the past integrated image and easily find, for example, a degraded part. The display control unit 122 may control the display unit 114 to display three or more integrated images side by side.

The display control unit 122 may control the display unit 114 to not only display the integrated image 701 and the integrated image 702 side by side but also highlight and display a degraded part detected by image processing or the like. Also, the display control unit 122 may control the display unit 114 to display a differential image in which a difference between the two integrated images is highlighted.

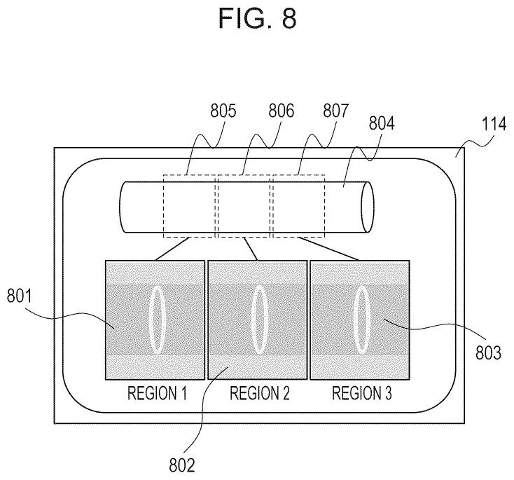

Although FIG. 7 illustrates the example in which the integrated images (the current integrated image 701 and the past integrated image 702) obtained by capturing images of a single region of a single object at different times are displayed side by side, the present embodiment is not limited thereto. For example, integrated images obtained by capturing images of a plurality of regions of a single object at different times may be displayed side by side. FIG. 8 illustrates an example of non-destructive instruction of piping. FIG. 8 illustrates an example in which integrated images 801, 802, and 803 captured at different times are displayed side by side. The integrated image 801 is obtained by capturing an image of a region 1 (805) of piping 804. The integrated image 802 is obtained by capturing an image of a region 2 (806) of the piping 804. The integrated image 803 is obtained by capturing an image of a region 3 (807) of the piping 804. The display control unit 122 may synthesize the integrated images 801 to 803 into a panoramic image and display the panoramic image, instead of displaying, side by side, the integrated images 801 to 803 captured at the different times. In a case where the integrated images are synthesized into the panoramic image, and the panoramic image is displayed, the operator can easily associate the plurality of regions of the object with the plurality of integrated images and can efficiently inspect the object.

The panoramic synthesis may be performed after the positions of the piping included in the integrated images 801 to 803 are aligned. In a case where the panoramic synthesis is performed after the alignment, a sense of incongruity of an image of a synthesized part is reduced and the operator will be able to efficiently check the image to be inspected. In addition, in a case where the adjacent integrated images during the panoramic synthesis include a common region, the accuracy of the panoramic synthesis can be improved. Therefore, the display control unit 122 may control the display unit 114 to display a display prompting the operator to capture an image including the common region.

In addition, the display control unit 122 may control the display unit 114 to display, side by side, an integrated image obtained by capturing an image of the piping from the front side of the piping and an integrated image obtained by capturing an image of the piping from the rear side of the piping. The display control unit 122 may synthesize the integrated images into a panoramic image and control the display unit 114 to display the panoramic image.

According to the present disclosure, it is possible to suppress the need for image re-capturing.

### Other Embodiments

Embodiment(s) of the present disclosure can also be realized by a computer of a system or apparatus that reads out and executes computer-executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer-executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer-executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has described exemplary embodiments, it is to be understood that some embodiments are not limited to the disclosed exemplary embodiments. The scope of the following claims is to be accorded the broadest interpretation so as to encompass all such modifications and equivalent structures and functions.

This application claims priority to Japanese Patent Application No. 2023-119162, which was filed on Jul. 21, 2023 and which is hereby incorporated by reference herein in its entirety.

What is claimed is:

1. An information processing apparatus comprising:
at least one memory storing instructions; and
at least one processor, wherein the at least one processor is configured to execute the instructions to:
generate a first integrated image by integrating a plurality of radiation images obtained by image capturing using a radiation generating apparatus that generates radiation and a radiation imaging apparatus, and generate a second integrated image different from the first integrated image using the plurality of radiation images and at least one radiation image obtained by the image capturing after the plurality of radiation images are obtained; and
control a display device to change a display based on a determination regarding an image quality of the first integrated image to a display based on a determination regarding an image quality of the second integrated image.

2. The information processing apparatus according to claim 1, wherein the at least one processor is further configured to execute the instructions to:
generate a third integrated image using the plurality of radiation images and the at least one radiation image used for generating the second integrated image, and at least one radiation image obtained by the image capturing after the at least one radiation image used for generating the second integrated image is obtained, and
further control the display device to change the display based on the determination regarding the image quality of the second integrated image to a display based on a determination regarding an image quality of the third integrated image.

3. The information processing apparatus according to claim 1, wherein
each of the displays based on the determinations is either a display prompting to end the image capturing or a display prompting to continue the image capturing.

4. The information processing apparatus according to claim 1, wherein the at least one processor is further configured to execute the instructions to:
control the display device to display, as a moving image, the plurality of radiation images obtained by the image capturing.

5. The information processing apparatus according to claim 1, wherein
each of the determinations regarding the image qualities is a determination of an SNR that is a ratio of a signal component to a noise component.

6. The information processing apparatus according to claim 5, wherein the at least one processor is further configured to execute the instructions to:
in a case where the SNR is determined to be greater than a predetermined value, control the display device to display at least one of the SNR and an integrated image used for determining the SNR.

7. The information processing apparatus according to claim 5, wherein the at least one processor is further configured to execute the instructions to:
in a case where the SNR is determined to be greater than a predetermined value, control the display device to display a display prompting to end the image capturing.

8. The information processing apparatus according to claim 5, wherein the at least one processor is further configured to execute the instructions to:
store the SNR and an integrated image used for determining the SNR to a storage device in a case where the SNR is determined to be greater than a predetermined value.

9. The information processing apparatus according to claim 5, wherein the at least one processor is further configured to execute the instructions to:
generate the second integrated image in a case where the SNR is determined to be smaller than a predetermined value.

10. The information processing apparatus according to claim 5, wherein the at least one processor is further configured to execute the instructions to:
control the display device to display the plurality of radiation images and the SNR of the plurality of radiation images.

11. The information processing apparatus according to claim 5, wherein the at least one processor is further configured to execute the instructions to:
control the display device to display a graph indicating a change in the SNR of the plurality of radiation images.

12. The information processing apparatus according to claim 11, wherein the at least one processor is further configured to execute the instructions to:
control the display device to display time information that is obtained using the change and indicates a time required until an integrated image having an SNR greater than a predetermined value is obtained.

13. The information processing apparatus according to claim 11, wherein the at least one processor is further configured to execute the instructions to:
control the display device to display a warning in a case where a change rate of the SNR obtained using the change is smaller than a first threshold.

14. The information processing apparatus according to claim 12, wherein the at least one processor is further configured to execute the instructions to:
control the display device to display a warning in a case where the time indicated by the time information is longer than a second threshold.

15. The information processing apparatus according to claim 1, wherein the at least one processor is further configured to execute the instructions to:
after the plurality of radiation images are obtained, generate the second integrated image every time at least one radiation image is obtained.

16. The information processing apparatus according to claim 1, wherein the at least one processor is further configured to execute the instructions to:
control the display device to display a fourth integrated image obtained at a different time from a time when the image capturing is performed.

17. An information processing apparatus comprising:
at least one memory storing instructions; and
at least one processor, wherein the at least one processor is configured to execute the instructions to:
generate a first integrated image by integrating a plurality of radiation images obtained by image capturing using a radiation generating apparatus that generates radiation and a radiation imaging apparatus, and generate a second integrated image different from the first integrated image using the plurality of radiation images and at least one radiation image obtained by the image capturing after the plurality of radiation images are obtained; and
output a signal based on a determination regarding an image quality of the second integrated image.

18. An information processing system comprising:
a radiation imaging apparatus; and
the information processing apparatus according to claim 1, wherein the information processing apparatus is communicably connected to the radiation imaging apparatus.

19. An information processing method comprising:
generating a first integrated image by integrating a plurality of radiation images obtained by image capturing using a radiation generating apparatus that generates radiation and a radiation imaging apparatus, and generating a second integrated image different from the first integrated image using the plurality of radiation images and at least one radiation image obtained by the image capturing after the plurality of radiation images are obtained; and
controlling a display device to change a display based on a determination regarding an image quality of the first integrated image to a display based on a determination regarding an image quality of the second integrated image.

20. A non-transitory computer-readable medium storing computer-executable instructions that, when executed by a computer, cause the computer to:
generate a first integrated image by integrating a plurality of radiation images obtained by image capturing using a radiation generating apparatus that generates radiation and a radiation imaging apparatus, and generate a second integrated image different from the first integrated image using the plurality of radiation images and at least one radiation image obtained by the image capturing after the plurality of radiation images are obtained; and
control a display device to change a display based on a determination regarding an image quality of the first integrated image to a display based on a determination regarding an image quality of the second integrated image.
